(19) United States
(12) Patent Application Publication
(10) Pub. No.: US 2022/0142581 A1
(43) Pub. Date: May 12, 2022

(54) PILLOW WITH INDEPENDENT-SUPPORTING STRUCTURE

(71) Applicant: SHUN-TIEN HUNG, Taichung City (TW)

(72) Inventor: SHUN-TIEN HUNG, Taichung City (TW)

(21) Appl. No.: 17/093,678

(22) Filed: Nov. 10, 2020

(51) Int. Cl. A61B 5/00; A47G 9/10

(52) U.S. Cl. CPC A61B 5/6892; A47G 9/1045; A61B 5/4806; A61B 2562/043; A61B 2562/0247; A61B 2562/164; A47G 2009/1018

(57) ABSTRACT

A pillow has an independent-supporting structure and a headrest cushion. The independent-supporting structure has a supporting plate. The supporting plate has multiple supporting elements disposed at intervals. Each one of the multiple supporting elements is able to be independently pressed, to independently restore, and to be independently deformed. The headrest cushion is resilient and is supported by the supporting plate for supporting a head of a human. When a user rests his head on the pillow, each supporting element is deformed independently to provide independent support to the cervical spine of the user.

### BACKGROUND OF THE INVENTION

### 1. Field of the Invention

The present invention relates to a pillow, and more particularly to a pillow having an independent-supporting structure.

### 2. Description of Related Art

Many people nowadays suffer from sleep disorder. People may be still hyperactive before going to bed and hard to fall asleep due to fast-paced life, stressful work, and drinks with caffeine. Moreover, people usually relieve stress by browsing information on cell phones before falling asleep. However, blue light emitted from the cell phones also disturbs secretion of melatonin that helps people to fall asleep.

In order to improve sleep quality, a conventional pillow with Marshall coils is provided. The conventional pillow with Marshall coils has multiple springs respectively disposed in multiple cotton pockets for supporting the cervical spine of a user to provide proper support to the cervical spine of the user and to resolve the problem of sleep disorder. However, the multiple springs of the conventional pillow with Marshall coils have a same coefficient of elasticity and cannot provide various supporting forces to different body parts of the user. For example, the user's cervical spine needs larger supporting force than the user's head. Nevertheless, under the restriction that the springs have the same coefficient of elasticity, the conventional pillow with Marshall coils cannot provide various supporting forces to the different body parts of the user.

To overcome the shortcomings of the conventional pillow with Marshall springs, the present invention provides a pillow with an independent-supporting structure to mitigate or obviate the aforementioned problems.

### SUMMARY OF THE INVENTION

The main objective of the present invention is to provide a pillow that provides independent support to the cervical spine of the user.

The pillow has an independent-supporting structure and a headrest cushion. The independent-supporting structure has a supporting plate. The supporting plate has multiple supporting elements disposed at intervals. Each one of the multiple supporting elements is able to be independently pressed, to independently restore, and to be independently deformed. The headrest cushion is resilient and is supported by the supporting plate for supporting a head of a human. When a user rests his head on the pillow, each supporting element is deformed independently to provide independent support to the cervical spine of the user.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of a first embodiment of a pillow in accordance with the present invention;

FIG. 2 is a perspective view of an independent-supporting structure of the pillow in FIG. 1;

FIG. 3 is a top view of the independent-supporting structure in FIG. 2;

FIG. 4 is an operational cross-sectional side view of the pillow in FIG. 1, showing a head of a human resting on the pillow;

FIG. 5 is another operational cross-sectional side view of the pillow in FIG. 1, showing a neck of the human resting on the pillow;

FIG. 6 is an enlarged cross-sectional side view of the pillow in FIG. 1, showing that the independent-supporting structure is pre-stressed and resiliently bended;

FIG. 7 is another enlarged cross-sectional side view of the pillow in FIG. 1, showing that the independent-supporting structure is disposed on a supporting cushion;

FIG. 8A is a partially cross-sectional perspective view of a second embodiment of a pillow in accordance with the present invention;

FIG. 8B is an enlarged cross-sectional perspective view of the pillow in FIG. 8A;

FIG. 9A is a partially cross-sectional perspective view of a third embodiment of a pillow in accordance with the present invention;

FIG. 9B is an enlarged cross-sectional perspective view of the pillow in FIG. 9A;

FIG. 10A is a partially cross-sectional perspective view of a fourth embodiment of a pillow in accordance with the present invention;

FIG. 10B is an enlarged cross-sectional perspective view of the pillow in FIG. 10A;

FIG. 11 is an exploded perspective view of a fifth embodiment of a pillow in accordance with the present invention;

FIG. 12 is an exploded perspective view of the independent-supporting structure and multiple flexible sensors of the pillow in FIG. 11;

FIG. 13 is a cross-sectional side view of the pillow in FIG. 11;

FIG. 14 is another cross-sectional side view of the pillow in FIG. 11, showing each flexible sensor being a pressure sensor;

FIG. 15 is a cross-sectional side view of a sixth embodiment of a pillow in accordance with the present invention, showing each flexible sensor being a flex sensor; and

FIG. 16 is an optional perspective view of the pillow in FIG. 11, showing the pillow and a portable device working together.

### DETAILED DESCRIPTION OF PREFERRED EMBODIMENTS

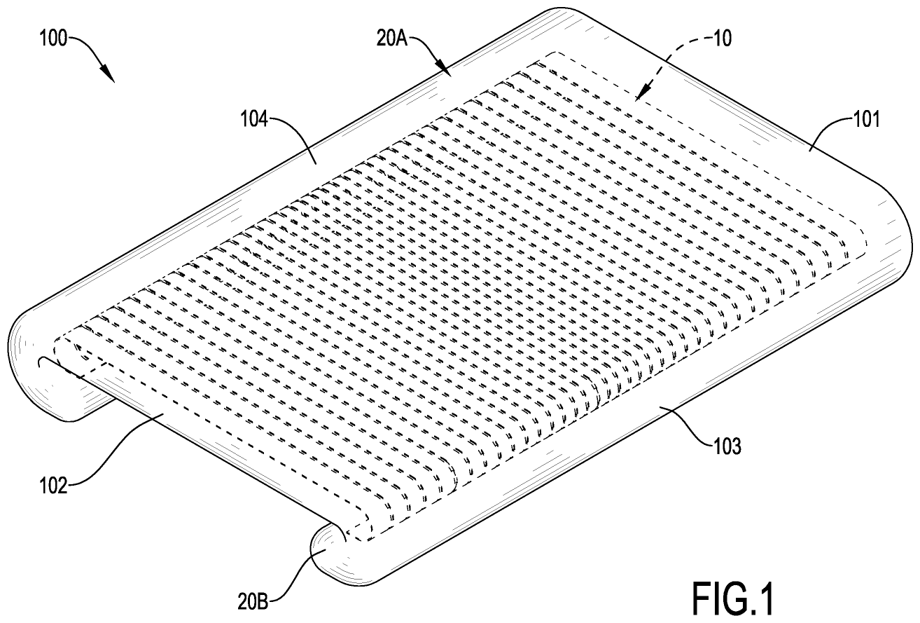

With reference to FIGS. 1 to 3, a pillow 100 in accordance with the present invention has a longitudinal direction, two ends 101, 102, two lateral sides 103, 104, an independent-supporting structure 10, a headrest cushion 20A, and a supporting cushion 20B. The two ends 101, 102 of the pillow 100 are opposite in the longitudinal direction of the pillow 100. The two lateral sides 103, 104 are parallel to the longitudinal direction of the pillow 100.

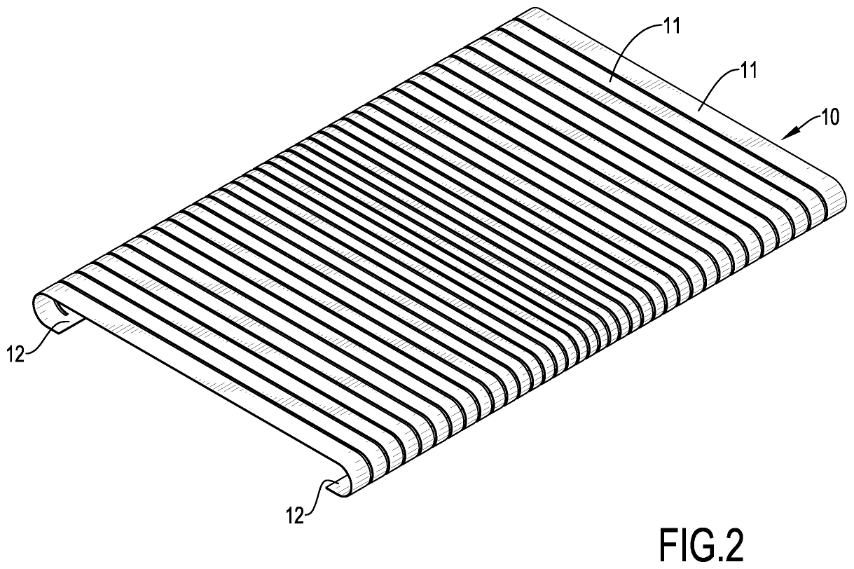

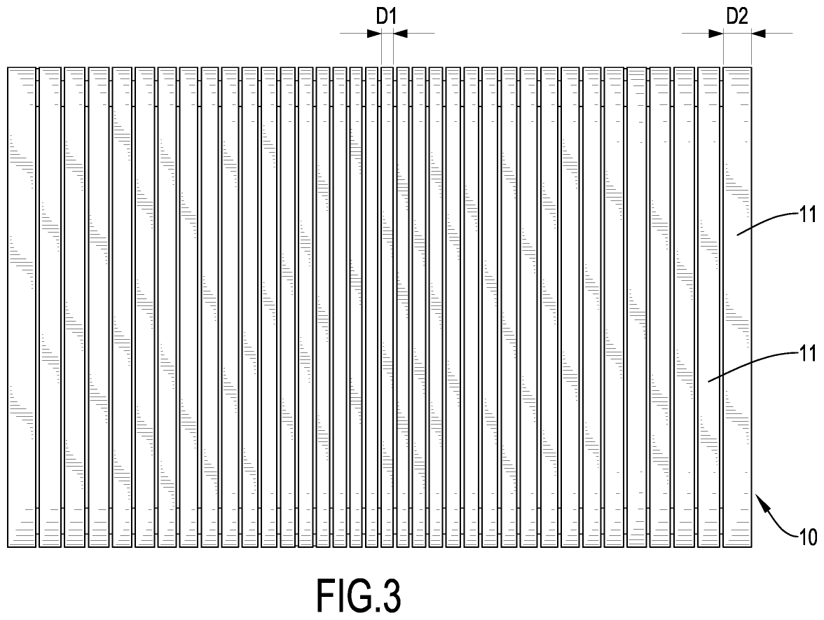

With reference to FIGS. 1 and 2, a first embodiment of the pillow 100 in accordance with the present invention is shown. In the first embodiment of the present invention, the independent-supporting structure 10 has a supporting plate and two connecting elements 12. The supporting plate has multiple supporting elements 11. Each one of the multiple supporting elements 11 is an elongated sheet and has two opposite ends respectively extending toward the two lateral sides 103, 104 of the pillow 100. The multiple supporting elements 11 are arranged at intervals and along the longitudinal direction of the pillow 100. The supporting elements 11 are independently deformable by being independently pressed and independently restoring. In the first embodiment of the present invention, each one of the multiple supporting elements 11 is elongated and has an elongating direction. The multiple supporting elements 11 are parallelly disposed. The elongating direction of each one of the multiple supporting elements 11 and the longitudinal direction of the pillow 100 are perpendicular. Each one of the multiple supporting elements 11 extends to the two lateral sides 103, 104 of the pillow 100. The multiple supporting elements 11 are parallel and arranged at intervals. Practically, each one of the multiple supporting elements 11 may be a ring, and the multiple supporting elements 11 are concentrically arranged at intervals. Arranged patterns of the multiple supporting elements 11 and outlines of each one of the multiple elements 11 are not restricted as long as each one of the multiple supporting elements 11 can be independently pressed, can be independently deformed, and can independently restore. With reference to FIGS. 1 and 3, each one of the supporting elements 11 has a width. Widths of the multiple supporting elements 11 gradually decrease from two opposite ends 101, 102 of the pillow 100 towards a middle section of the pillow 100. For example, in FIG. 3, one of the multiple supporting elements 11 disposed at the left end of the pillow 100 has a width D2 of 18 mm, another one of the multiple supporting elements 11 disposed at the middle section of the pillow 100 has a width D1 of 10 mm.

The two connecting element 12 are connected to the multiple supporting elements 11, are pre-stressed, and are disposed oppositely in the elongating direction of each one of the multiple supporting elements 11. When the pillow 100 in accordance with the present invention is placed in a bed, the two connecting elements 12 are configured to support the pillow 100.

Practically, the independent-supporting structure 10 may have one connecting element 12 only. The connecting element 12 and the multiple supporting elements 11 surround a receiving space for receiving cell phones, portable chargers, or audio players. The connecting element 12 may have an extending arm being parallel to each one of the multiple supporting elements 11 to form an opening disposed at one of the two sides 103, 104 of the pillow 100. Structure of the connecting element 12 can be designed according to various needs and for requirements of different visual effects. The structure of each connecting element 12 is not restricted as long as each one of the multiple supporting elements 11 can be independently pressed, can be independently deformed, and can independently restore.

In the present invention, the independent-supporting structure 10 is made of a composite material with thermoplastic matrix. Practically, the independent-supporting structure 10 may be made of metal, plastic, or even wood as long as each one of the multiple supporting elements 11 can be independently pressed, can be independently deformed, and can independently restore. The material of the independent-supporting structure 10 is not restricted.

In the first embodiment of the present invention, a prepreg is bended at two sides of the prepreg. Then, multiple slits are defined through the prepreg to form the multiple supporting elements 11 and the two connecting elements 12 respectively connected to the two opposite ends of each one of the multiple supporting elements 11.

In the first embodiment of the present invention, the independent-supporting structure 10 is composed by a carbon fiber composite layer and two glass fiber composite layers. The carbon fiber composite layer is clamped between the two glass fiber composite layers having better tenacity than carbon fiber composite layer, so the independent-supporting structure 10 has good ductility to sustain loads and is less breakable.

Moreover, when carbon fibers of the carbon fiber composite layer are broken, the two glass fiber composite layers wrap the carbon fiber composite layer that is broken to avoid exposure of the broken carbon fibers. Practically, the independent-supporting structure 10 may be implemented as a carbon fiber composite layer covered by a glass fiber composite layer or even be implemented as a carbon fiber composite layer according to practical requirements.

With reference to FIGS. 1 and 2, the headrest cushion 20A is designed for a head of a user to rest thereon, is resilient, and is supported by the supporting plate of the independent-supporting structure 10. The headrest cushion 20A may be made of foam, rubber, emulsion, or similar flexible materials. In the first embodiment of the present invention, the headrest cushion 20A and the supporting cushion 20B are integrally connected and fully wrapped around the independent-supporting structure 10. Practically, the pillow 100 may merely have the headrest cushion 20A covering the independent-supporting structure 10, that is, the pillow 100 only has the headrest cushion 20A disposed at a portion for the user's head to rest.

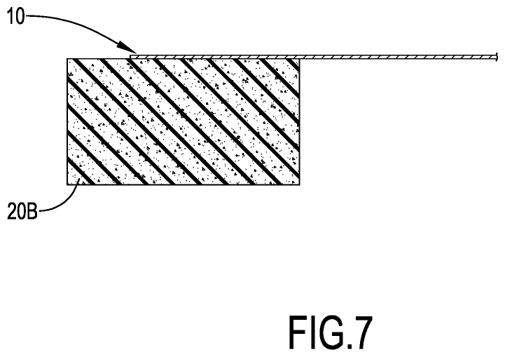

With reference to FIG. 7, the independent-supporting structure 10 may even omit the connecting elements 12, and the multiple supporting elements 11 are directly connected to the supporting cushion 20B. In addition, the pillow 100 in accordance with the present invention may further have a pillow case made of cloth. The pillow case may be sleeved on the headrest cushion 20A and the supporting cushion 20B.

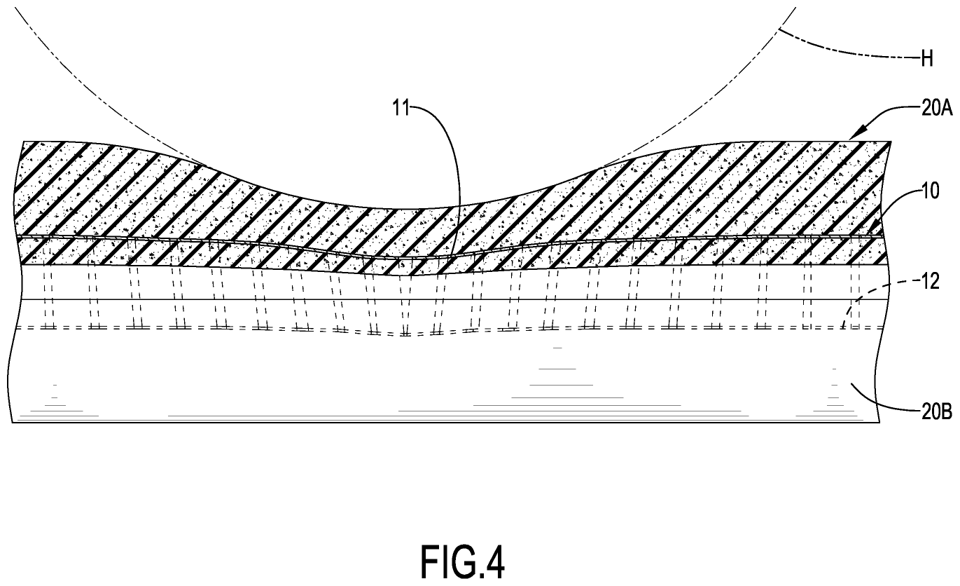

With reference to FIG. 4, the user's head H is resting on the middle section of the pillow 100 in accordance with the present invention. The widths of the multiple supporting elements 11 gradually decrease from two opposite ends 101, 102 of the pillow 100 towards a middle section of the pillow 100 as mentioned above. Therefore, the supporting elements 11 disposed at the middle section of the pillow 100 are subjected to maximum pressure and have maximum deformations accordingly. And, each one of the multiple supporting elements 11 is connected to the two connecting elements 12 via the two opposite ends of the supporting element 11. Middle sections of the supporting elements 11 disposed at the middle section of the pillow 100 can be expanded toward the two opposite ends 101, 102 of the pillow 100 by being subjected to the pressure. Therefore, the supporting elements 11 disposed at the middle section of the pillow 100 can wrap the user's head H and can support the user's head H laterally.

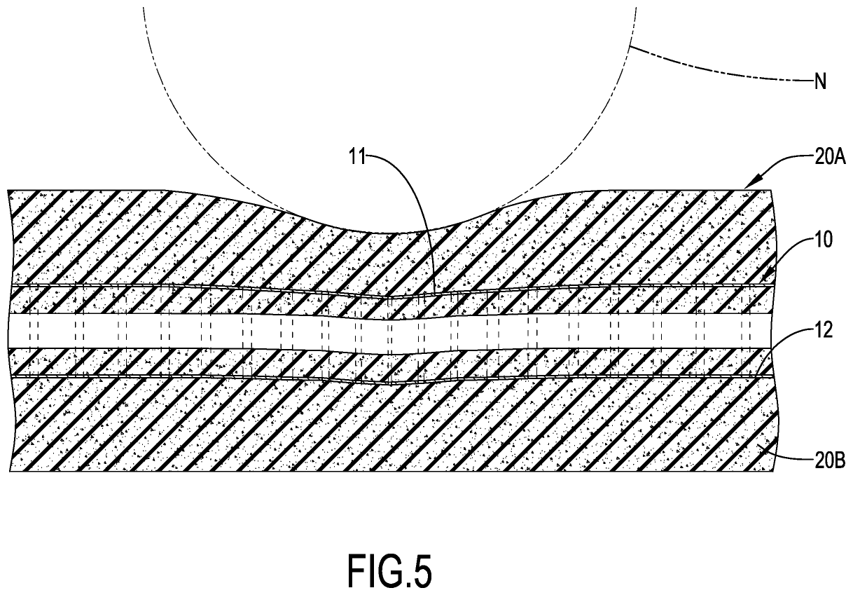

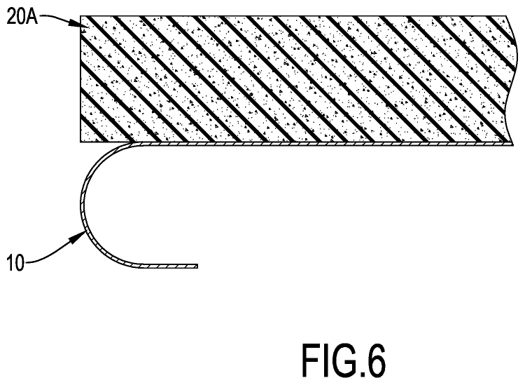

With reference to FIG. 5, when the user's head is resting on the pillow 100 in accordance with the present invention, a neck of the user is resting on one of the two lateral sides 103, 104 of the pillow 100. Since the two opposite ends of each supporting element 11 are respectively connected to the two connecting elements 12, portions respectively disposed adjacent to the two opposite ends of each supporting element 11 are difficult to be expanded. Therefore, either of the lateral sides 103, 104 can provide sufficient support to the user's neck. Moreover, since the supporting cushion 20B is disposed beneath the two connecting elements 12 and supports the two connecting elements 12, weight of the user's neck is transmitted via the headrest cushion 20A, the independent-supporting structure 10, and to the supporting cushion 20B. The supporting cushion 20B being resilient and deformable provides a space for deformation and depression to either connecting element 12. Therefore, the portions of the supporting elements 11 disposed adjacent to either of the lateral sides 103, 104 can be concentrated as being subjected to the weight of the user's neck and enhance support to the user's neck.

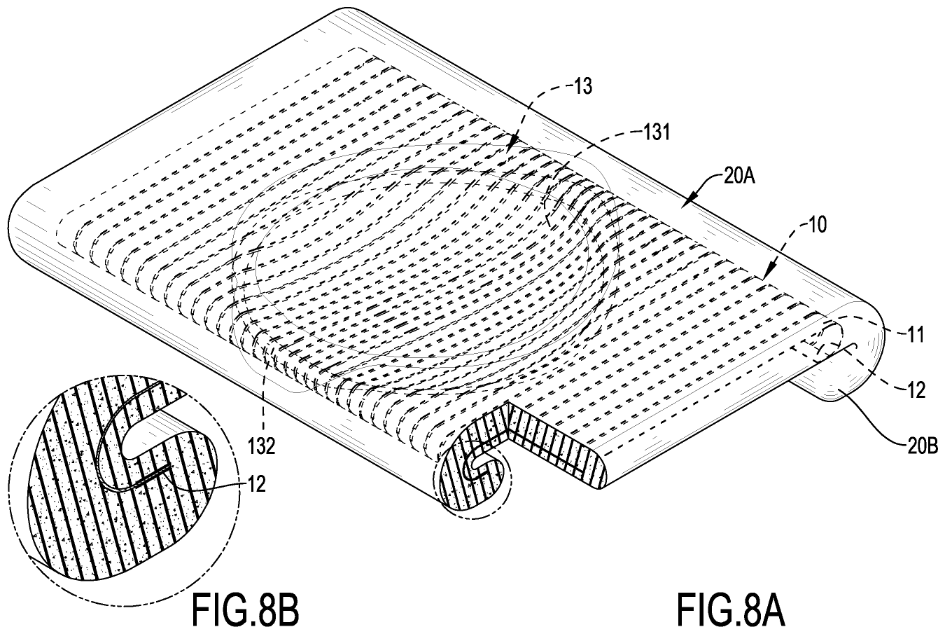

With further reference to FIGS. 8A and 8B, a second embodiment of the present invention is shown. The second embodiment is similar to the first embodiment. In the second embodiment, the pillow 100 also has the independent-supporting structure 10, the headrest cushion 20A and the supporting cushion 20B with the headrest cushion 20A and the supporting cushion 20B being wrapped around and covering the independent-supporting structure 10. In the second embodiment of the present invention, the independent-supporting structure 10 further has a recess 13. The recess 13 is concaved on the supporting elements 11 disposed at the middle section of the pillow 100 in accordance with the present invention. The recess 13 has a headrest area 131 and a neck-rest area 132. The headrest area 131 is disposed at the middle section of the supporting elements 11 disposed at the middle section of the pillow 100. The headrest cushion 20A is recessed according to position of the headrest area 131. The neck-rest area 132 is disposed adjacent to one of the two opposite ends of each supporting element 11 disposed at the middle section of the pillow 100. The neck-rest area 132 is disposed at one of the two lateral sides 103, 104 of the pillow 100.

Furthermore, in the second embodiment, the independent-supporting structure 10 is made of composite materials with thermoplastic matrix. Each one of the two connecting elements 12 has two sheets. One of the two opposite ends of each supporting element 11 is clamped between the two sheets of one of the two connecting elements 12. The other one of the two opposite ends of the supporting element 11 is clamped between the two sheets of the other one of the two connecting elements 12. Each supporting element 11 is connected to the two connecting elements 12 via hot pressing. Practically, the two connecting elements 12 may be made of different materials.

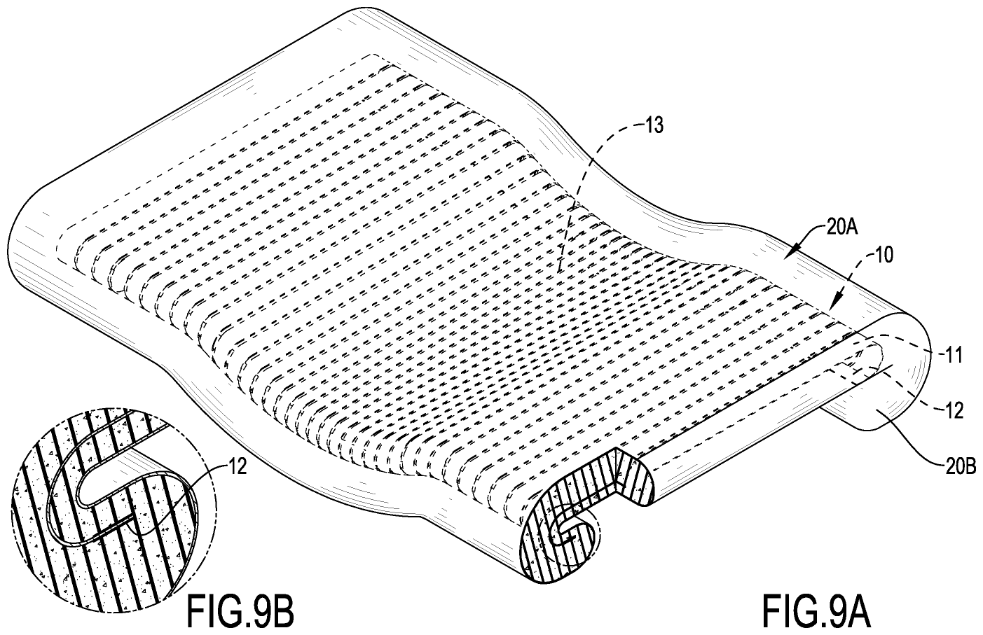

With further reference to FIGS. 9A and 9B, a third embodiment of the present invention is shown. The third embodiment and the second embodiment are similar. In the third embodiment, the pillow 100 also has the independent-supporting structure 10, the headrest cushion 20A and the supporting cushion 20B with the headrest cushion 20A and the supporting cushion 20B being wrapped around and covering the independent-supporting structure 10. In the third embodiment of the present invention, the recess 13 of the independent-supporting structure 10 is also recessed on the supporting elements 11 disposed at the middle section of the pillow 100 in accordance with the present invention. In the third embodiment, the recess 13 extends to the two lateral sides 103, 104 of the pillow 100.

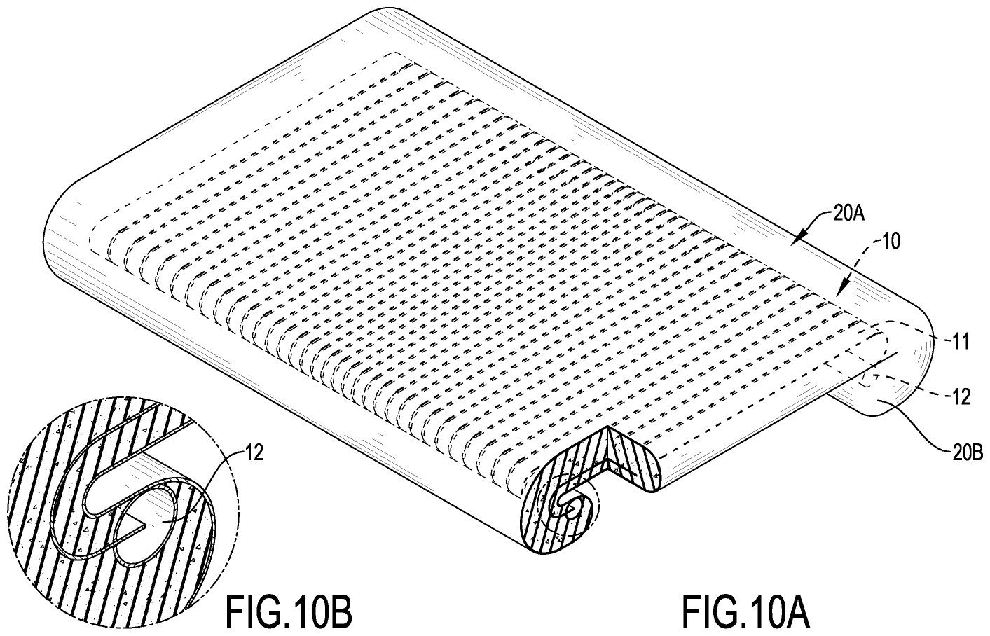

With further reference to FIGS. 10A and 10B, a fourth embodiment of the present invention is shown. The fourth embodiment and the first embodiment are similar. In the fourth embodiment, the pillow 100 also has the independent-supporting structure 10, the headrest cushion 20A and the supporting cushion 20B with the headrest cushion 20A and the supporting cushion 20B being wrapped around and covering the independent-supporting structure 10. In the fourth embodiment, each one of the two connecting elements 12 is a tube with a peripheral wall. The two opposite ends of each supporting element 11 are respectively inserted through the peripheral walls of the two connecting elements 12.

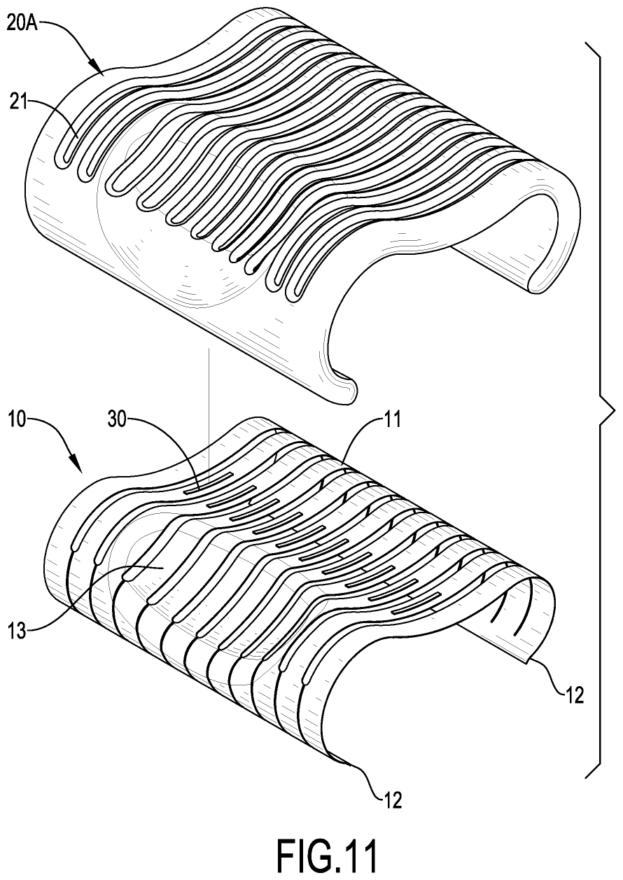

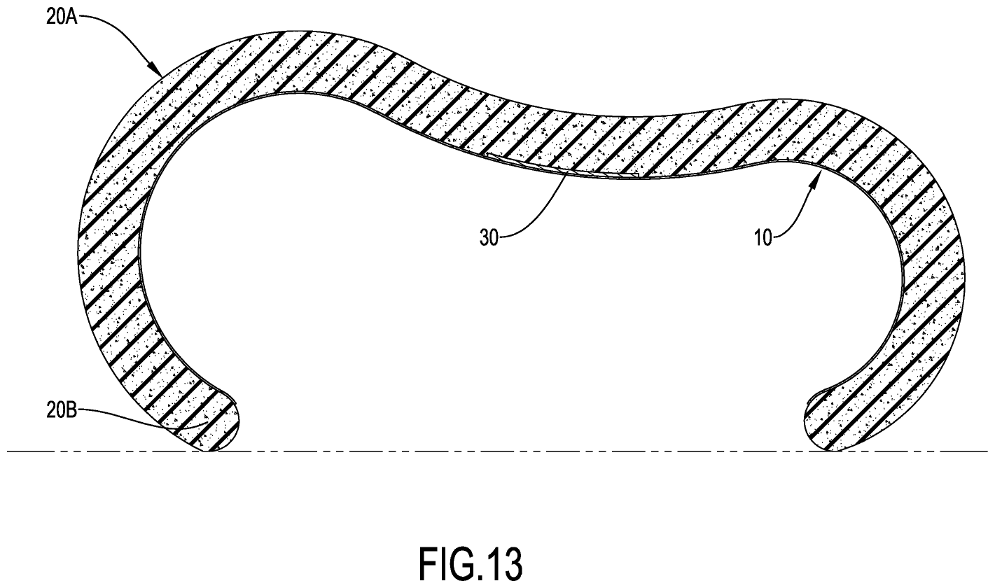

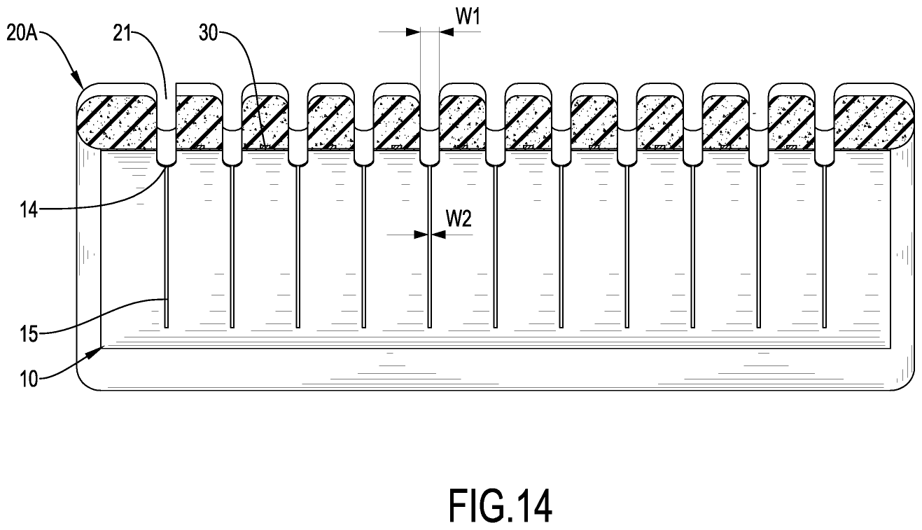

With further reference to FIGS. 11 to 13, a fifth embodiment of the present invention is shown. The fifth embodiment and the second embodiment are similar.

In the fifth embodiment, the pillow 100 also has the independent-supporting structure 10, the headrest cushion 20A and the supporting cushion 20B with the headrest cushion 20A and the supporting cushion 20B being wrapped around and covering the independent-supporting structure 10. And the independent-supporting structure 10 also has the recess 13. In the fifth embodiment, the pillow 100 further has multiple flexible sensors 30. In the fifth embodiment, the recess 13 is disposed at one of the two lateral sides 103, 104 of the pillow 100. The supporting plate of the independent-supporting structure 10 has multiple through holes 14 and multiple slits 15. Each one of the through holes 14 is formed between two of the supporting elements 11 that are disposed adjacent to each other, is elongated, and is parallel to the multiple supporting elements 11. Each two of the slits 15 are formed between two of the supporting elements 11 that are disposed adjacent to each other, and the two slits respectively extend from two opposite ends of a corresponding one of the through holes toward opposite directions. Each slit 15 has a width being narrower than a width of each through hole 14.

In the fifth embodiment, the headrest cushion 20A has multiple vents 21. The multiple vents 21 are respectively aligned with the through holes 14 and respectively communicate with the through holes 14 for providing good ventilation and convection to the head of the user.

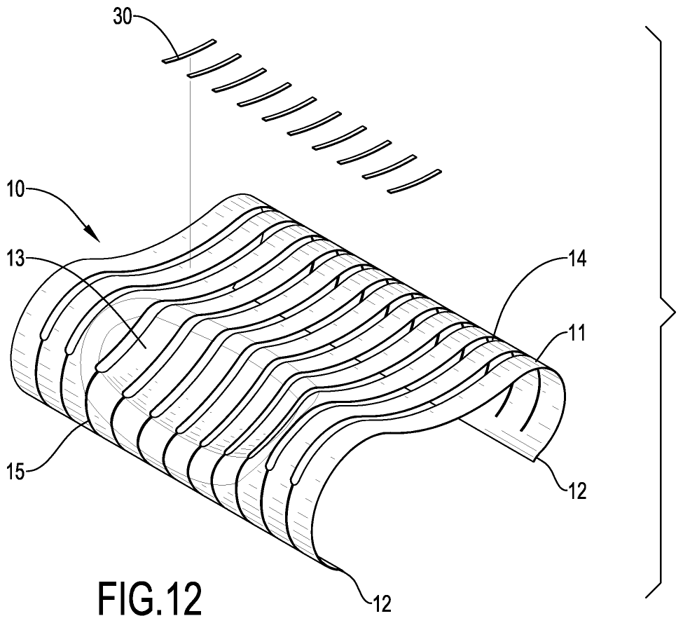

With reference to FIGS. 11 and 12, each flexible sensor 30 includes a flexible printed circuit. The multiple flexible sensors 30 are respectively mounted to the multiple supporting elements 11 and are arranged in an array. The multiple flexible sensors 30 can fully capture data regarding contact between the user and the pillow 100 in accordance with the present invention.

Each flexible sensor 30 may be a pressure sensor and is disposed on a top face of a corresponding one of the supporting elements 11. When the user's head and neck are resting on the headrest cushion 20A, pressure caused by weight of the user's head and neck is transmitted to each flexible sensor 30. Then, each sensor 30 obtains data of the pressure.

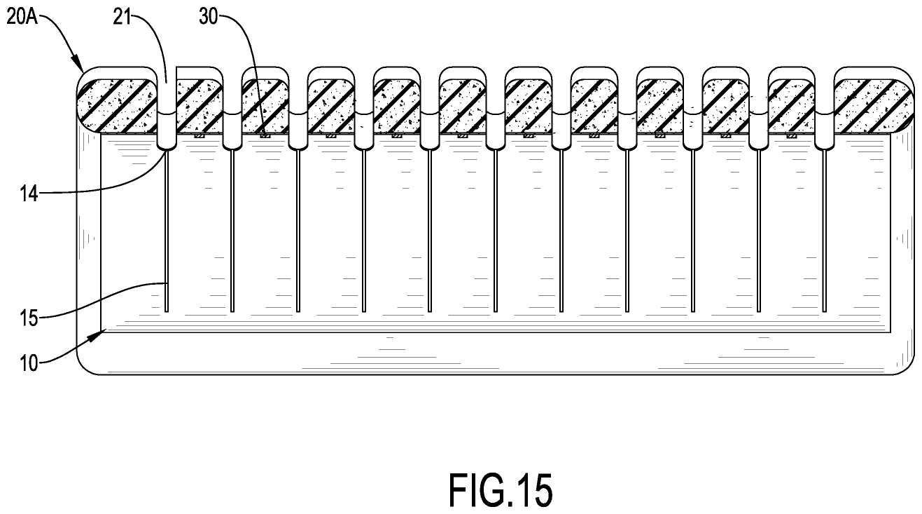

With further reference to FIG. 15, in a sixth embodiment of the present invention, each flexible sensor 30 may be a flex sensor and is disposed on a bottom face of a corresponding one of the supporting elements 11. When the pressure caused by the weight of the user's head and neck is subjected to the headrest cushion 20A, the pressure is transmitted to each supporting element 11. Each supporting element 11 subjected to the pressure is deformed. A corresponding one of the flexible sensors 30 attached to the supporting element 11 can detect deformation of the supporting element 11 and obtain the data of the pressure.

In the present invention, each supporting element 11 can be independently pressed and independently restore. Therefore, the pillow 100 in accordance with the present invention provides function of independent support similar to Marshall coils. With reference to FIGS. 4 and 5, when the user's head is resting on the pillow 100, each supporting element 11 can be independently pressed, independently restore, and generate deformations of different degrees. Therefore, the pillow 100 in accordance with the present invention provides independent support.

The through hole 14 disposed between each two adjacent supporting elements 11 communicates with a corresponding one of the vents 21, which not only provides ventilation and convection, but also promotes degree of deformation of each supporting element 11. Therefore, the supporting elements 11 produce different deformations according to contour of the user's head and provide sufficient support to the user's neck. Compared to the conventional pillow with Marshall coils that is difficult to be equipped with sensors, the top face and the bottom face of each supporting element 11 can be conveniently equipped with the flexible sensor 30 to obtain the data of contact between the user and the pillow 100.

In the fifth embodiment, the two slits 15 disposed between each two adjacent supporting elements 11 further promote the degree of deformation of each supporting element 11. Each supporting element 11 can rapidly and agilely deform according to the pressure generated by the user's head and neck. The data obtained by the multiple flexible sensors 30 is distinguishable and reliable. Particularly, when each flexible sensor 30 is a flex sensor, the two slits 15 disposed between each two adjacent supporting elements 11 facilitate each supporting element 11 to deform with ease and facilitate a corresponding flexible sensor 30 to obtain data.

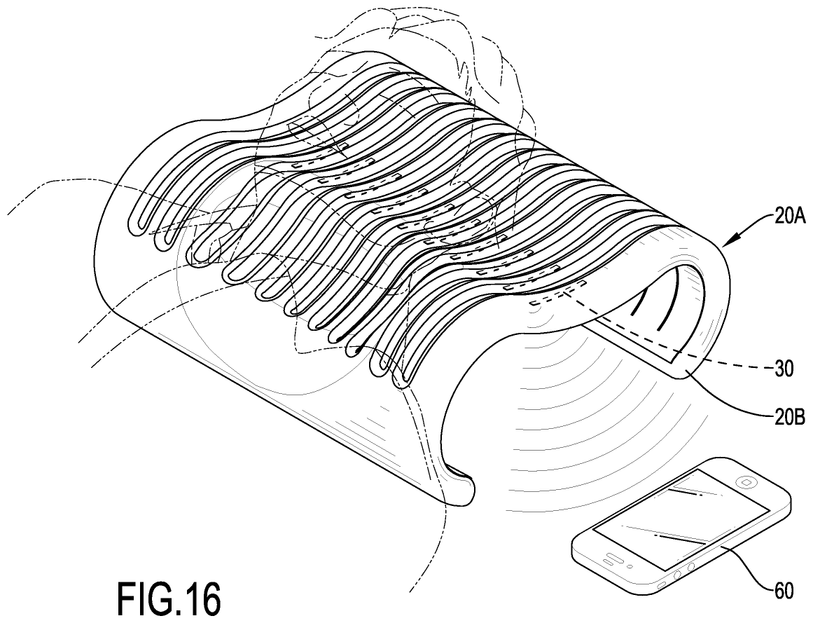

With reference to FIG. 16, the multiple flexible sensors 30 can obtain quantifiable data and transmit the quantifiable data to a cell phone 60 or the internet via wired or wireless transmission. The quantifiable data is computed to define behavior patterns of the users. For instance, the quantifiable data can be used to determine whether the user lies on his back or side. The user can change his/her behavior patterns to improve sleep quality. The data collected by the multiple flexible sensors 30 in a long term can be used as a basis for observing shift of the user's behavior patterns.

In addition, though the independent-supporting structure 10, the headrest cushion 20A, the supporting cushion 20B, and the flexible sensors 30 are applied to pillows, these features may be also applied to mattresses, seat cushions, or back cushions. Arranging patterns and structures of the multiple supporting elements 11 may be designed according to different products.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A pillow comprising:
an independent-supporting structure having a supporting plate, the supporting plate having multiple supporting elements disposed at intervals, and the supporting elements being independently deformable by being independently pressed and independently restoring; and
a headrest cushion being resilient and supported by the supporting plate for supporting a head of a human.

2. The pillow as claimed in claim 1, wherein each one of the multiple supporting elements is elongated, and the multiple supporting elements are parallelly disposed.

3. The pillow as claimed in claim 2, wherein
the independent-supporting structure has a connecting element;
one of two opposite ends of each one of the multiple supporting elements is connected to the connecting element.

4. The pillow as claimed in claim 2, wherein
the independent-supporting structure has two connecting elements;
two opposite ends of each one of the multiple supporting elements are respectively connected to the two connecting elements.

5. The pillow as claimed in claim 4, wherein
the independent-supporting structure is made of a composite material with thermoplastic matrix;
each one of the two connecting elements has two sheets;
one of the two opposite ends of each one of the multiple supporting elements is clamped between the two sheets of one of the two connecting elements; and
the other one of the two opposite ends of each one of the multiple supporting elements is clamped between the two sheets of the other one of the two connecting elements.

6. The pillow as claimed in claim 4, wherein
each one of the two connecting elements is a tube with a peripheral wall; and
the two opposite ends of each one of the multiple supporting elements are inserted through the peripheral walls of the two connecting elements respectively.

7. The pillow as claimed in claim 3, wherein widths of the multiple supporting elements gradually decrease from two opposite ends of the pillow toward a middle section of the pillow.

8. The pillow as claimed in claim 5, wherein widths of the multiple supporting elements gradually decrease from two opposite ends of the pillow toward a middle section of the pillow.

9. The pillow as claimed in claim 6, wherein widths of the multiple supporting elements gradually decrease from two opposite ends of the pillow toward a middle section of the pillow.

10. The pillow as claimed in claim 8, wherein the pillow has a supporting cushion disposed below the independent-supporting structure.

11. The pillow as claimed in claim 9, wherein the pillow has a supporting cushion disposed below the independent-supporting structure.

12. The pillow as claimed in claim 10, wherein the pillow has multiple flexible sensors respectively mounted to the multiple supporting elements, and each one of the multiple flexible sensors is a flexible circuit.

13. The pillow as claimed in claim 11, wherein the pillow has multiple flexible sensors respectively mounted to the multiple supporting elements, and each one of the multiple flexible sensors is a flexible circuit.

14. The pillow as claimed in claim 12, wherein
each one of the multiple flexible sensors is a pressure sensor; and
the multiple flexible sensors are disposed between the headrest cushion and the multiple supporting elements.

15. The pillow as claimed in claim 13, wherein
each one of the multiple flexible sensors is a pressure sensor; and
the multiple flexible sensors are disposed between the headrest cushion and the multiple supporting elements.

16. The pillow as claimed in claim 12, wherein
each one of the multiple flexible sensors is a flex sensor; and
the multiple supporting elements are disposed between the headrest cushion and the multiple flexible sensors.

17. The pillow as claimed in claim 13, wherein
each one of the multiple flexible sensors is a flex sensor; and
the multiple supporting elements are disposed between the headrest cushion and any one of the multiple flexible sensors.

18. The pillow as claimed in claim 2, wherein
the supporting plate of the independent-supporting structure has multiple through holes, and each one of the through holes is formed between two of the multiple supporting elements that are disposed adjacent to each other, is elongated, and is parallel to the multiple supporting elements.

19. The pillow as claimed in claim 18, wherein
the supporting plate of the independent-supporting structure has multiple slits, each two of the slits are formed between two of the multiple supporting elements that are disposed adjacent to each other, and the two slits respectively extend from two opposite ends of a corresponding one of the through holes toward opposite directions; and
each slit has a width being narrower than a width of each through hole.

20. The pillow as claimed in claim 19, wherein each one of the multiple supporting elements is pre-stressed and resiliently bended.
